(19) United States
(12) Patent Application Publication
(10) Pub. No.: US 2022/0213028 A1
(43) Pub. Date: Jul. 7, 2022

(54) METHOD FOR THE PURIFICATION OF ISOCYANATES

(71) Applicant: Covestro Intellectual Property GmbH & Co. KG, Leverkusen (DE)

(72) Inventors: Christoph Eggert, Köln (DE); Thomas Fischer, Köln (DE); Ulrich Geron, Köln (DE); Ulrich Esser, Kürten (DE)

(21) Appl. No.: 17/609,154

(22) PCT Filed: Apr. 30, 2020
(86) PCT No.: PCT/EP2020/062015

§ 371 Date: Nov. 5, 2021

(30) Foreign Application Priority Data
May 7, 2019 (EP) 19172870.8

(51) Int. Cl. C07C 263/20

(52) U.S. Cl. CPC C07C 263/20

(57) ABSTRACT

The invention relates to a process for purifying at least one isocyanate selected from the group consisting of aliphatic, cycloaliphatic, araliphatic isocyanates and mixtures thereof, to a process for producing at least one isocyanate selected from the group consisting of aliphatic, cycloaliphatic, araliphatic isocyanates and mixtures thereof, to the isocyanate obtainable by this process, to a polyurethane constructed from at least one such isocyanate, to the use of this isocyanate and to an optical component containing at least one polyurethane according to the invention.

The invention relates to a process for purifying at least one isocyanate selected from the group consisting of aliphatic, cycloaliphatic, araliphatic isocyanates and mixtures thereof, to a process for producing at least one isocyanate selected from the group consisting of aliphatic, cycloaliphatic, araliphatic isocyanates and mixtures thereof, to the isocyanate obtainable by this process, to a polyurethane constructed from at least one such isocyanate, to the use of this isocyanate and to an optical component containing at least one polyurethane according to the invention.

Aliphatic, cycloaliphatic or araliphatic diisocyanates are typically produced by phosgenation of the corresponding diamines Irrespective of the precise procedure the reaction product is typically obtained as a mixture of monomeric and polymeric species. This crude process product is worked up in a manner known to those skilled in the art, wherein the workup generally comprises a multistage vacuum distillation and affords the monomeric diisocyanate in high purity. Such diisocyanates are suitable for the production of lightfast, i.e. non-yellowing, polyurethanes.

If optical materials, such as optical lenses, are to be produced, it is generally not sufficient to use lightfast polyurethanes. In addition, elevated demands are placed on the clarity of the material, i.e. the turbidity values must be very low.

DE 1805957 and U.S. Pat. No. 3,658,656 describe for example processes for purifying xylylene diisocyanate (XDI) produced from xylylenediamine (XDA) and phosgene. To this end the XDI obtained from the reaction is first distilled in the presence of an inert gas under vacuum to separate off low-boiling impurities. XDI together with higher molecular weight residue accumulates in the bottom of the distillation apparatus. In a further distillation stage this XDI is separated from the bottom product as distillate. The distillation is carried out for example at a pressure of 5 to 200 mmHg (667 Pa to 26 665 Pa) at the column top and a temperature of 170° C. to 185° C. in the column bottom.

While the lightfast diisocyanates produced in this way from aliphatic, cycloaliphatic or araliphatic amines have a high purity, for example in terms of chlorinated by-products, it has been found that they have a propensity for turbidity and do not always meet the relevant requirements for use in optical materials.

Despite the lower reactivity of the aliphatically, cycloaliphatically or araliphatically bonded NCO groups compared to aromatically bonded NCO groups, a di- or oligomerization of the diisocyanates may surprisingly occur. For the very reactive aromatic diphenylmethane diisocyanates, for example, GB 1413074 describes the formation of uretdione dimers in the distillate, which can largely be avoided by rapidly cooling the distillate. However, the attempt to transfer this technical teaching, for example to xylylene diisocyanate. showed that turbidity cannot be adequately avoided in this way.

WO 2007/051740 A1 describes a process for producing mixtures containing diphenylmethane diisocyanate and its higher molecular weight homologues. It is described that a polyisocyanate mixture containing diphenylmethane diisocyanate (2-ring MDI) and triphenylmethane diisocyanate (3-ring MDI) is converted at room temperature into a biphasic mixture having a liquid phase and a solid crystalline phase. The liquid phase may be separated therefrom by simple decanting or filtering of the liquid phase to adjust the ratio of 2-ring to 3-ring MDI. The conditions of the possible filtration are not discussed further and, due to the diisocyanates used, neither a lightfast nor a turbidity-free diisocyanate is obtained.

Proceeding from this prior art, it is an object of the present invention to remedy at least one, preferably two or more, of the abovementioned disadvantages of the prior art. It is a particular object of the present invention to provide a process for purifying at least one aliphatic, cycloaliphatic or araliphatic diisocyanates which makes it possible to adjust the turbidity of the corresponding diisocyanate to a value such that polyurethanes and/or polythiourethanes employable in optical applications, in particular as optical lenses, are obtained. It is a further object of the present invention to provide a process which makes it possible to obtain the recited diisocyanates in sufficiently high optical quality, in particular with a very low turbidity.

These objects are achieved by the process according to the invention for purifying at least one isocyanate selected from the group consisting of aliphatic, cycloaliphatic, araliphatic isocyanates and mixtures thereof, comprising at least the steps of:
(A) providing the at least one isocyanate,
(B1) filtering the at least one isocyanate from step (A) through a filter having a permeability of 5 to 1001/(m²·min) under standard conditions, Δp=1 bar, H₂O, 20° C., to obtain the at least one purified isocyanate,
   or comprising at least the steps of:
(A) providing the at least one isocyanate,
(B2) filtering the at least one isocyanate from step (A) through a filter having a permeability of 5 to 1000 l/(m²·min) under standard conditions, Δp=1 bar, H₂O, 20° C., and
(C) filtering the at least one isocyanate through a filter having a maximum pore size of 0.02 to 10 μm,
   wherein the sequence of steps may be (A), (B2), (C) or (A), (C), (B2), to obtain the at least one purified isocyanate.

The individual steps of the process according to the invention are described in detail hereinbelow.

Step (A) of the process according to the invention comprises providing the at least one isocyanate.

According to the invention any isocyanate known to those skilled in the art selected from the group consisting of aliphatic, cycloaliphatic, araliphatic isocyanates and mixtures thereof may be used in step (A). The isocyanate employed according to the invention is especially selected from the group of aliphatic, cycloaliphatic, araliphatic diisocyanates and mixtures thereof.

Aliphatic diisocyanates particularly preferably employed according to the invention are for example selected from the group consisting of hexamethylene diisocyanate (HDI), pentamethylene diisocyanate (PDI), 1,4-butane diisocyanate, 1,8-diisocyanatooctane, 1,9-diisocyanatononane, 1,10-diisocyanatodecane, 1,11-diisocyanatoundecane, 1,12-diisocyanatododecane, 2-methylpentamethylene diisocyanate, 2,2-dimethylpentamethylene diisocyanate, neopentane diisocyanate, 2,2,4-trimethylhexamethylene diisocyanate and 2,4,4-trimethylhexamethylene diisocyanate and mixtures thereof. Very particularly preferably employed aliphatic diisocyanates are selected from the group consisting of HDI and PDI.

Cycloaliphatic diisocyanates particularly preferably employed according to the invention are for example selected from the group consisting of 3-isocyanatomethyl-3,5,5-trimethylcyclohexyl isocyanate (IPDI), 2,4′-diisocyanatodicyclohexylmethane (2,4′-H12-MDI), 4,4′-diisocyanatodicyclohexylmethane (4,4′-H12-MDI), 2,4′-methylene-bis(cyclohexyl) diisocyanate, 4,4′-diisocyanato-3,3′-dimethyldicyclohexylmethane, 2,4- and 2,6-diisocyanatomethylcyclohexane (H6TDI), 1,3-cyclohexane diisocyanate, 1,4-cyclohexane diisocyanate, 1,3-diisocyanato-2-methylcyclohexane, 1,3-diisocyanato-4-methyl-cyclohexane, 1,4-diisocyanato-3,3,5-trimethylcyclohexane, 1,3-bis(isocyanatomethyl)cyclohexane (H6XDI), 1,4-bis(isocyanatomethyl)cyclohexane, the isomers of bis(isocyanatomethyl)bicyclo[2.2.1]heptane (NBDI), in particular 2,5-bis(isocyanatomethyl)bicyclo[2.2.1]heptane (2,5-NBDI) or 2,6-bis(isocyanatomethyl)bicyclo[2.2.1]heptane (2,6-NBDI), and mixtures thereof.

Araliphatic diisocyanates particularly preferably employed according to the invention are for example selected from the group consisting of xylylene diisocyanate (XDI), in particular 1,3-xylylene diisocyanate (m-XDI) or 1,4-xylylene diisocyanate (p-XDI), 1,3-bis(1-isocyanato-1-methylethyl)benzene (m-TMXDI), 1,4-bis(1-isocyanato-1-methylethyl)benzene (p-TMXDI) and mixtures thereof. Meta-xylylene diisocyanate is very particularly preferred. Meta-xylylene diisocyanate (1) and para-xylylene diisocyanate (2) are shown below:

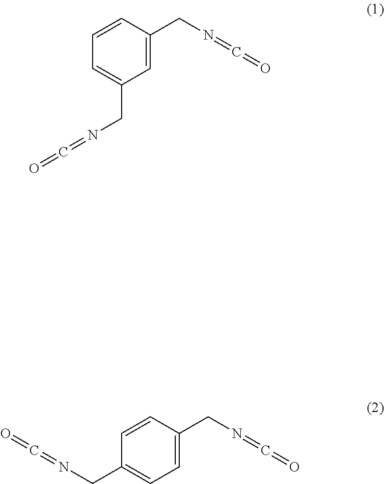 [SMILES: O=C=NCc1cccc(CN=C=O)c1 ; O=C=NCc1ccc(CN=C=O)cc1]

It is very particularly preferable when in step (A) of the process according to the invention the isocyanate is selected from the group consisting of bis(isocyanatomethyl)bicyclo[2.2.1]heptane (NBDI), in particular 2,5-bis(isocyanatomethyl)bicyclo[2.2.1]heptane (2,5-NBDI) or 2,6-bis(isocyanatomethyl)bicyclo[2.2.1]heptane (2,6-NBDI), pentamethylene diisocyanate, 1,3-bis(isocyanatomethyl)cyclohexane (H6XDI), xylylene diisocyanate, in particular meta-xylylene diisocyanate and/or para-xylylene diisocyanate, and mixtures thereof.

The providing of the at least one isocyanate, in particular the at least one diisocyanate, in step (A) of the process according to the invention comprises in particular producing the at least one isocyanate and preferably also a first purification to obtain the product which is then purified according to the invention in steps (B1) or (B2) and (C).

Step (A) of the process according to the invention in particular comprises producing the at least one isocyanate by phosgenation of the corresponding at least one amine with phosgene.

For the particularly preferred case where according to the invention bis(isocyanatomethyl)bicyclo[2.2.1]heptane (NBDI), in particular 2,5-bis(isocyanatomethyl)bicyclo[2.2.1]heptane (2,5-NBDI) or 2,6-bis(isocyanatomethyl)bicyclo[2.2.1]heptane (2,6-NBDI), pentamethylene diisocyanate, 1,3-bis(isocyanatomethyl)cyclohexane (H6XDI), xylylene diisocyanate, in particular meta-xylylene diisocyanate and/or para-xylylene diisocyanate, or mixtures thereof are purified/produced, it is preferable to employ bis(aminomethyl)bicyclo[2.2.1]heptane (NBDA), in particular 2,5-bis(aminomethyl)bicyclo[2.2.1]heptane (2,5-NBDA) or 2,6-bis(aminomethyl)bicyclo[2.2.1]heptane (2,6-NBDA), pentamethylenediamine, 1,3-bis(aminomethyl)cyclohexane (H6XDA), xylylenediamine, especially meta-xylylenediamine and/or para xylylenediamine, or mixtures thereof as starting amines in the phosgenation.

For the very particularly preferred case where xylylene diisocyanate, in particular m-xylylene diisocyanate, is purified/produced according to the invention, it is preferable to employ xylylenediamine, in particular m-xylylenediamine, as the starting amine in the phosgenation.

The production of the at least one isocyanate by phosgenation of the at least one amine may be carried out in various ways.

In one embodiment the phosgenation of the at least one amine is carried out in the gas phase. To this end the amine is preferably evaporated and heated to a temperature within the temperature range from 200° C. to 600° C. Optionally the evaporation and also the use of the amine vapors generated in the evaporation is effected in the presence of an inert gas and/or of vapours of an inert solvent. The inert gas is preferably nitrogen. Suitable inert solvents are for example chlorobenzene, o-dichlorobenzene, xylene, chloronaphthalene or mixtures thereof.

The phosgene used in the phosgenation is preferably used in excess based on the amine. An amount of phosgene corresponding to 150% to 350% of theory based on the proceeding phosgenation reaction is generally sufficient. The phosgene stream is preferably heated to a temperature in the range from 200° C. to 600° C. prior to the reaction.

To perform the phosgenation the preheated, amine-containing stream and the likewise preheated phosgene stream are preferably continuously passed into a cylindrical reaction space and mixed with one another therein. Suitable cylindrical reaction spaces are for example tubular reactors which generally consist of steel, glass, alloyed or enameled steel. They generally have a length which is sufficient to allow complete reaction of the amine with the phosgene under the process conditions.

The dimensions of the reaction space are preferably selected such that a turbulent flow having a Reynolds number of at least 2500 prevails in the reaction space. This is generally ensured when the flow rate is more than 90 m/s. Such a flow rate may be ensured by adjusting an appropriate differential pressure between the product conduits to the reaction space and the exit from the reaction space. In general the pressure in the feed conduits is 200 to 300 mbar(g) and at the exit from the reaction space is 150 to 200 mbar(g).

Upon termination of the phosgenation reaction in the reaction space the mixture continuously exiting the reaction space is preferably freed of the isocyanate formed. This may be effected for example by selective condensation in an inert solvent such as for example chlorobenzene or dichlorobenzene. If the amine-containing stream already contained an inert solvent it is preferable to use the same solvent here. The temperature of the solvent is preferably chosen such that on the one hand it is above the decomposition temperature of the carbamide acid chloride corresponding to the isocyanate and on the other hand the isocyanate condenses or dissolves in the solvent while phosgene, hydrogen chloride and any inert gas pass through the condensation stage in gaseous form. Solvent temperatures in the range from 120° C. to 200° C. are particularly suitable.

The gas mixture passing through the condensation stage to recover the at least one isocyanate is then preferably freed of excess phosgene in a manner known per se. This can be effected by means of a cold trap, absorption in an inert solvent (for example chlorobenzene, MCB, or dichlorobenzene, ODB) maintained at a temperature of −10° C. to 8° C., or adsorption and hydrolysis on activated carbon. The hydrogen chloride gas passing through the phosgene recovery stage may preferably be recycled in a manner known per se to recover the chlorine required for phosgene synthesis.

In a further embodiment the phosgenation of the at least one amine is carried out in the liquid phase. The reaction may then be performed in various ways. Either the amine is directly reacted with phosgene in an inert liquid medium (base phosgenation) or the amine is initially converted to the corresponding salt by reaction with hydrogen chloride gas or carbon dioxide in an inert liquid medium and then reacted with phosgene (hydrochloride or carbamate phosgenation). Suitable liquid media for all phosgenations include in particular chlorobenzene and/or dichlorobenzene.

In base phosgenation the reaction is performed in two stages in the inert liquid medium. Such reactions are described for example in W. Siefken, Liebigs Annalen der Chemie, 562 (1949) p. 96. In the first stage, the cold phosgenation, the temperature of the reaction mixture is preferably maintained in a range between 0° C. and 100° C. This forms a suspension which contains carbamic acid chloride, amine hydrochloride and small amounts of free isocyanate. It is preferable to initially charge a solution of phosgene in an inert solvent and then add a solution or suspension of the amine in the same solvent and optionally further phosgene. This keeps concentration of free amine low and thus inhibits undesired formation of ureas.

In the second stage, the hot phosgenation, the temperature is increased and is preferably in a range from 120° C. to 200° C. It is maintained in this range while further phosgene is supplied until the reaction to afford the isocyanate has terminated, i.e. the evolution of HCl comes to a halt. Phosgene is advantageously used in excess. If required, the reaction may be performed with introduction of an inert gas both in the cold phosgenation and in the hot phosgenation.

In the amine hydrochloride or carbamate phosgenation, the amine is preferably initially reacted with hydrogen chloride gas or carbon dioxide in an inert liquid medium to produce the corresponding salt. The reaction temperature during this salt formation is preferably in a range from 0° C. 80° C. The phosgenation step follows as a second step which is substantially similar to the hot phosgenation from the abovedescribed base phosgenation. Here too, the temperature is therefore preferably kept in the range from 120° C. to 200° C. while phosgene and optionally an inert gas are introduced into the reaction mixture. The introduction is carried out until the reaction to form the isocyanate has terminated. Here too, phosgene is preferably employed in excess to accelerate the reaction.

After termination of the reaction, both in the base phosgenation and in the amine hydrochloride or carbamate phosgenation, the remaining phosgene and hydrogen chloride gas are preferably blown out with an inert gas, preferably with nitrogen. If necessary, a filtration may be carried out to remove any solids such as unreacted amine hydrochlorides.

The thus obtained at least one isocyanate is preferably worked up by distillation before it is subjected to further processing in the steps (B1) or (B2) in accordance with the invention. This distillative workup in particular frees the at least one isocyanate from the employed solvent, chlorinated byproducts and higher boiling residues.

The present invention thus especially relates to the process according to the invention, wherein step (A) comprises distilling the at least one isocyanate, in particular before the steps (B1) or (B2).

The distilling preferably performed in step (A) of the process according to the invention may be carried out in a manner known to those skilled in the art. Since production of the isocyanates typically employs solvents having a lower boiling point than the respective isocyanate the distillation generally comprises a solvent separation. This distillation step may also comprise separating low-boiling secondary components, in particular chlorinated low-boiling secondary components. Optionally after further purification steps the solvent may be reused in the reaction for producing the isocyanate, preferably diisocyanate.

Such a purification process further comprises a purifying distillation for separating the isocyanate from high-boiling residue.

All distillation steps are preferably carried out under vacuum to reduce the temperatures required for distillation and thus the thermal stress on the product. In particular the distillation steps are performed at pressures of 1 to 500 mbar(a) and a bottoms temperature of 90° C. to 250° C., preferably 120° C. to 190° C., particularly preferably 120° C. to 170° C. Especially in the purifying distillation the pressure is preferably in the range from 1 to 100 mbar(a), particularly preferably in the range from 5 to 50 mbar(a), and the bottoms temperature is in the range from 120° C. to 185° C., very particularly preferably in the range from 120° C. to 170° C.

In order, where required, to inhibit the formation of uretdiones in the distillate the cooling of the distillate is carried out as quickly as possible.

Step (A) of the process according to the invention preferably affords the at least one isocyanate, in particular meta-xylylene diisocyanate and/or para-xylylene diisocyanate, in pure form, i.e. in a purity of ≥99.0%, preferably of ≥99.5% and particularly preferably of ≥99.7%.

However, the at least one isocyanate obtained in step (A) has a turbidity of for example more than 0.5 NTU, preferably 0.5 to 40 NTU, in each case determined according to DIN EN ISO 7027-1: 2016-11. The at least one isocyanate provided in step (A) according to the invention may be transferred into step (B1) or (B2)/(C) without further intermediate steps.

However, in a preferred embodiment the isocyanate is subjected to a ripening after step (A) and preferably before step (B1) or (B2). To this end the isocyanate is stored preferably at a temperature in the range from 0° C. to 100° C., particularly preferably in the range from 10° C. to 80° C., very particularly preferably in the range from 15° C. to 55° C. The isocyanate may optionally be stirred during storage to avoid settling of any solids at the bottom of the storage container. The ripening is preferably carried out over a period of 1 to 500 h, particularly preferably over a period of 2 bis 100 h. Precursor substances dissolved in the isocyanate, i.e. substances that may contribute to the formation of turbidity in the isocyanate, can therefore be bound and thus more easily removed in the subsequent filtration step. Such a procedure has an advantageous effect on the storage stability of the isocyanate.

According to the invention the at least one isocyanate provided in step (A) may in a first alternative be transferred to step (B1) and filtered, i.e. the sequence of steps for this embodiment is (A) followed by (B1). In a second embodiment the at least one isocyanate provided in step (A) may be transferred to steps (B2) and (C) and filtered, i.e. the sequence of steps for this embodiment is (A) followed by (B2) followed by (C), or (A) followed by (C) followed by (B2), preferably (A) followed by (B2) followed by (C). In all embodiments according to the invention the at least one isocyanate is obtained with the advantages according to the invention, i.e. especially a low turbidity of less than 0.35 NTU.

Step (B1) of the process according to the invention comprises filtering the at least one isocyanate from step (A) through a filter having a permeability of 5 to 1001/(m²·min) under standard conditions, Δp=1 bar, H₂O, 20° C., to obtain the at least one purified isocyanate.

In step (B1) of the process according to the invention the at least one isocyanate obtained in step (A) is filtered through a filter having a permeability of 5 to 100 l/(m²·min), preferably 5 to 60 l/(m²·min), particularly preferably 5 to 401/(m²·min), in each case under standard conditions, Δp=1 bar, H₂O, 20° C.

According to the invention step (B1) may be carried out at any temperature appearing suitable to those skilled in the art. Step (B1) is preferably carried out at a temperature of 5° C. to 190° C., particularly preferably 5° C. to 100° C., very particularly preferably 5° C. to 60° C.

According to the invention step (B1) may be carried out at any pressure appearing suitable to those skilled in the art. Step (B1) is preferably carried out at a pressure of 0.1 to 7 bar(a), particularly preferably 0.5 to 6 bar(a), very particularly preferably 1 to 4 bar(a), in each case measured on the inflow side of the filter.

Apart from the abovementioned permeability, the filter employed in step (B1) of the process according to the invention is not subject to any further restrictions. The filter employed in step (B1) preferably contains at least one material selected from natural fibers, synthetic polymers, perlite, kieselguhr and mixtures thereof. It is further preferable according to the invention when a filter layer made of the recited material is employed. Suitable apparatuses for accommodating the corresponding filter layer are known per se to those skilled in the art.

Step (B1) of the process according to the invention is further preferably operated at a specific filtrate flow of 50 to 1500 kg/(m²·h), preferably 100 to 1000 kg/(m²·h), particularly preferably 200 to 500 kg/(m²·h).

It is likewise further preferable when in step (B1) the pressure drop over the filter is 0.01 to 5 bar, preferably 0.1 to 3 bar and particularly preferably 0.5 to 2 bar, very particularly preferably 1 to 1.5 bar.

After step (B1) of the process according to the invention the at least one purified isocyanate, preferably xylylene diisocyanate, especially preferably meta-xylylene diisocyanate and/or para-xylylene diisocyanate, has a turbidity of less than 0.35 NTU, particularly preferably 0.05 to 0.35 NTU, very particularly preferably 0.10 to 0.32 NTU, in each case determined according to DIN EN ISO 7027-1: 2016.

Due to the particularly low turbidity obtained by the process according to the invention comprising the steps (A) and (B1) the at least one purified isocyanate, optionally after one or more intermediate steps, may preferably be employed in a further process step for synthesis of polyurethanes and/or polythiourethanes for optical applications.

In a second embodiment of the process according to the invention the at least one isocyanate provided in step (A) is transferred to step (B2).

Step (B2) of the process according to the invention relates to filtering the at least one isocyanate from step (A) through a filter having a permeability of 5 to 1000 l/(m²·min) under standard conditions, Δp=1 bar, H₂O, 20° C.

In step (B2) of the process according to the invention the at least one isocyanate obtained in step (A) is filtered through a filter having a permeability of 5 to 10001/(m²·min), preferably 5 to 9501/(m²·min), particularly preferably 80 to 9801/(m²·min), in each case under standard conditions, Δp=1 bar, H₂O, 20° C.

According to the invention step (B2) may be carried out at any temperature appearing suitable to those skilled in the art. Step (B2) is preferably carried out at a temperature of 5° C. to 190° C., particularly preferably 5° C. to 100° C., very particularly preferably 5° C. to 60° C.

According to the invention step (B2) may be carried out at any pressure appearing suitable to those skilled in the art. Step (B2) is preferably carried out at a pressure of 0.1 to 7 bar(a), particularly preferably 0.5 to 6 bar(a), very particularly preferably 1 to 4 bar(a), in each case measured on the inflow side of the filter.

Apart from the abovementioned permeability, the filter employed in step (B2) of the process according to the invention is not subject to any further restrictions. The filter employed in step (B2) preferably contains at least one material selected from natural fibers, synthetic polymers, perlite, kieselguhr and mixtures thereof. It is further preferable according to the invention when a filter layer made of the recited material is employed. Suitable apparatuses for accommodating the corresponding filter layer are known per se to those skilled in the art, for example modular filters.

Step (B2) of the process according to the invention is further preferably operated at a specific filtrate flow of 50 to 1500 kg/(m²·h), preferably 100 to 1000 kg/(m²·h), particularly preferably 500 to 800 kg/(m²·h).

It is likewise further preferable when in step (B2) the pressure drop over the filter is 0.01 to 5 bar, preferably 0.1 to 3 bar.

The at least one isocyanate obtained in step (B2) of the process according to the invention is preferably transferred to step (C). The present invention thus preferably relates to the process according to the invention, wherein the steps are performed in the sequence (A) followed by (B2) followed by (C).

It is likewise possible, but less preferred, according to the invention for the at least one isocyanate provided in step (A) to be processed initially in step (C) and subsequently in step (B2).

Step (C) of the process according to the invention comprises filtering the at least one isocyanate through a filter having a maximum pore size of 0.02 to 10 μm.

In step (C) of the process according to the invention, the at least one isocyanate obtained in step (A) or (B2) is filtered through a filter having a maximum pore size of 0.02 to 10 μm, preferably 0.05 to 1 μm, particularly preferably 0.1 to 0.5 μm, very particularly preferably 0.15 to 0.3 μm.

In the context of the present invention the maximum pore size of the employed filters is determined according to ASTM F316-03 2011 for flexible filters, for example membrane filters, and according to ASTM E218-99 2011 for rigid filters, for example glass or ceramic filters.

According to the invention step (C) may be carried out at any temperature appearing suitable to those skilled in the art. Step (C) is preferably carried out at a temperature of 5° C. to 190° C., particularly preferably 5° C. to 100° C., very particularly preferably 5° C. to 60° C.

The present invention therefore preferably relates to the process according to the invention, wherein the steps (B1), (B2) and (C) are each carried out at a temperature of 5° C. to 190° C.

According to the invention step (C) may be carried out at any pressure appearing suitable to those skilled in the art. Step (C) is preferably carried out at a pressure of 0.1 to 7 bar(a), particularly preferably 0.5 to 6 bar(a), very particularly preferably 1 to 4 bar(a), in each case measured on the inflow side of the filter.

Apart from the abovementioned maximum pore size, the filter employed in step (C) of the process according to the invention is not subject to any further restrictions. The filter employed in step (C) preferably contains at least one material selected from natural fibers, synthetic polymers, glass, ceramic, metal and mixtures thereof.

According to the invention it is further preferable when step (C) employs a filter, for example a candle filter, disc filter, plate and frame filter, layer filter, membrane cartridges, made of the recited material. Suitable apparatuses for accommodating the corresponding filter are known per se to those skilled in the art.

Step (C) of the process according to the invention is further preferably operated at a specific filtrate flow of 20 to 1000 kg/(m²th), preferably 50 to 600 kg/(m²·h), particularly preferably 100 to 500 kg/(m²·h).

It is likewise further preferable when in step (B1) the pressure drop over the filter is 0.01 to 5 bar, preferably 0.1 to 3 bar and particularly preferably 0.5 to 1.5 bar.

The filtering steps step (B1), step (B2) and step (C) may be performed independently of one another in various ways.

It is possible for example to perform the filtering step as a so-called dead-end filtration. Here, the fluid stream to be filtered is conveyed against the filter and the only outlet for the fluid is through the filter. This forms a filtercake or at least a concentration gradient of the particles to be separated on the upstream side of the filter, thus increasing the filtration resistance. If the filtration resistance becomes excessively high backwashing with the filtrate makes it possible to loosen or remove the filtercake, thus reducing the filtration resistance again. In this procedure the pressure difference is preferably kept low to avoid compaction of the filtercake.

Alternatively, the fluid stream to be filtered may be passed along the filter in a tangential flow filtration, i.e. there is at least one inlet and at least one outlet for the fluid to be filtered on the upstream side of the filter. Depending on the outflow direction of the filtrate this may be referred to as a countercurrent filtration, i.e. the filtrate flows in the opposite direction to the feed stream, a cross-stream filtration, i.e. the filtrate flows perpendicularly to the feed stream, or a cocurrent filtration, i.e. the filtrate flows in the same direction as the feed stream. The filtration is referred to as a mixed flow filtration when the fluid to be filtered is agitated on the upstream side of the filter. The retentate can either be circulated on the upstream side of the filter or recirculated into the process elsewhere. Discarding of the retentate is inadvisable for economic reasons in tangential flow filtration. Advantages of tangential flow filtration are the avoidance of a filtercake and the minimization of the filtration resistance, but the conveying of the fluids entails higher energy and apparatus costs.

It is preferable when the filtering steps (B1) and (B2) and step (C) are carried out as a dead-end filtration. The reason for this is the high energy efficiency since recirculation streams are avoided. The retentate may preferably ultimately be disposed of together with the filter material.

In a preferred embodiment of the process according to the invention the filtration is carried out as a depth filtration. Advantages of such a depth filtration are a long service life of the filter and good separation even of difficult-to-filter particles. It is particularly preferable when the depth filter has an asymmetrical structure, i.e. the pores of the filter become finer from the inflow side with increasing penetration depth into the filter layer.

According to the invention performing the process comprising the steps (A) followed by (B2) followed by (C) or the process comprising the steps (A) followed by (C) followed by (B2) affords the at least one purified isocyanate, preferably xylylene diisocyanate, especially preferably meta-xylylene diisocyanate and/or para-xylylene diisocyanate having a turbidity of less than 0.35 NTU, particularly preferably 0.05 to 0.35 NTU, very particularly preferably 0.10 to 0.32 NTU, in each case determined according to DIN EN ISO 7027-1: 2016.

Due to this particularly low turbidity obtained according to the invention the at least one purified isocyanate, optionally after one or more intermediate steps, may preferably be employed in a further process step for synthesis of polyurethanes and/or polythiourethanes for optical applications.

The present invention further relates to a process for producing at least one isocyanate selected from the group consisting of aliphatic, cycloaliphatic, araliphatic isocyanates and mixtures thereof, preferably at least one diisocyanate selected from the group consisting of aliphatic, cycloaliphatic, araliphatic diisocyanates and mixtures thereof, comprising at least the steps of:
(A1) providing at least one amine corresponding to the isocyanate to be produced, preferably providing at least one diamine corresponding to the diisocyanate to be produced,
(A2) reacting the at least one amine from step (A1) with phosgene or a derivative thereof to obtain the at least one isocyanate, preferably reacting at least one diamine from step (A1) with phosgene or a derivative thereof to obtain the at least one diisocyanate, and
(A3) purifying the at least one isocyanate from step (A2), preferably purifying the at least one diisocyanate from step (A2),
   wherein step (A3) comprises at least the step of:
(B1) filtering the at least one isocyanate from step (A2) through a filter having a permeability of 5 to 1001/(m²·min) under standard conditions, Δp=1 bar, H₂O, 20° C., to obtain the at least one isocyanate, preferably filtering the at least one diisocyanate from step (A2) through a filter having a permeability of 5 to 100 l/(m²·min) under standard conditions, Δp=1 bar, H₂O, 20° C., to obtain the at least one diisocyanate,
   or that step (A3) comprises at least the steps of:
(B2) filtering the at least one isocyanate from step (A2) through a filter having a permeability of 5 to 10001/(m²·min) under standard conditions, Δp=1 bar, H₂O, 20° C., preferably filtering the at least one diisocyanate from step (A2) through a filter having a permeability of 5 to 10001/(m²·min) under standard conditions, Δp=1 bar, H₂O, 20° C., and
(C) filtering the at least one isocyanate through a filter having a maximum pore size of 0.02 to 10 μm, preferably filtering the at least one diisocyanate through a filter having a maximum pore size of 0.02 to 10 μm, wherein the sequence of steps may be (A1), (A2) and (B1) or (A1), (A2), (B2) and (C) or (A1), (A2), (C) and (B2), to obtain the at least one isocyanate, preferably the at least one diisocyanate.

Step (A1) comprises providing at least one amine corresponding to the isocyanate to be produced. According to the invention at least one amine selected from the group consisting of aliphatic, cycloaliphatic, araliphatic amines and mixtures thereof is accordingly used in step (A1). It is especially preferable to employ in step (A1) at least one diamine selected from the group consisting of aliphatic, cycloaliphatic, araliphatic diamines and mixtures thereof. Processes for producing corresponding amines and diamines are known per se to those skilled in the art. Technically relevant processes are for example the hydrogenation of the corresponding nitro compounds, the hydrogenation of the corresponding nitriles or the reductive amination of carbonyl compounds.

Diisocyanates suitable and preferred according to the invention are recited hereinabove and also apply to step (A1).

For the particularly preferred case where according to the invention bis(isocyanatomethyl)bicyclo[2.2.1]heptane (NBDI), in particular 2,5-bis(isocyanatomethyl)bicyclo[2.2.1]heptane (2,5-NBDI) or 2,6-bis(isocyanatomethyl)bicyclo[2.2.1]heptane (2,6-NBDI), 1,5-pentamethylene diisocyanate, 1,3-bis(isocyanatomethyl)cyclohexane (H₆XDI), xylylene diisocyanate, in particular meta-xylylene diisocyanate and/or para-xylylene diisocyanate, or mixtures thereof are produced, it is preferable to employ bis(aminomethyl)bicyclo[2.2.1]heptane (NBDA), in particular 2,5-bis(aminomethyl)bicyclo[2.2.1]heptane (2,5-NBDA) or 2,6-bis(aminomethyl)bicyclo[2.2.1]heptane (2,6-NBDA), 1,5-pentamethylenediamine, 1,3-bis(aminomethyl)cyclohexane (H₆XDA), xylylenediamine, especially meta-xylylenediamine and/or para xylylenediamine, or mixtures thereof in step (A1).

Step (A2) of the process according to the invention comprises reacting the at least one amine from step (A1) with phosgene or a derivative thereof to obtain the at least one isocyanate.

Step (A2) of the process according to the invention may generally be carried out according to any processes known to those skilled in the art. In a preferred embodiment the present invention relates to the process, wherein step (A2) is carried out in the gas phase or in the liquid phase, particularly preferably in the liquid phase.

The phosgenation of the corresponding at least one amine is especially carried out according to the invention when the at least one amine is reacted with phosgene in the liquid phase. Two-stage phosgenation processes, such as cold-hot phosgenation, amine hydrochloride phosgenation or carbamate phosgenation are particularly suitable. Amine hydrochloride phosgenation is very particularly suitable since it proceeds with high selectivity even in the case of very reactive amines such as for example XDA.

The present invention thus especially relates to the process according to the invention, wherein step (A2) is generally carried out at a temperature of 5° C. to 500° C. and/or a pressure of 0.5 to 10 bar(a).

When step (A2), i.e. the reacting of the at least one amine from step (A1) with phosgene or a derivative thereof to obtain the at least one isocyanate, is carried out in the gas phase the reaction temperature is preferably in the range from 300° C. to 500° C. and the pressure in the range from 0.5 to 3 bar(a).

Carrying out step (A2) as a base phosgenation in the liquid phase comprises performing initially a cold phosgenation, preferably at a temperature in the range from 0° C. to 100° C., particularly preferably in the range from 10° C. to 60° C., and subsequently a hot phosgenation at elevated temperature, preferably a temperature in the range from 120° C. to 200° C. The pressure is preferably 1 to 10 bar(a), particularly preferably 1.2 to 5 bar(a).

When step (A2) is carried out by the hydrochloride or carbamate route the temperature during the phosgenation reaction is preferably in the range from 80° C. to 200° C., particularly preferably in the range from 120° C. to 200° C. and very particularly preferably in the range from 120° C. to 180° C. The pressure is preferably 1 to 10 bar (a), particularly preferably 1.2 to 5 bar (a).

Further details and preferred embodiments of the phosgenation are described hereinabove.

Step (A3) of the process according to the invention comprises purifying the isocyanate from step (A2), wherein step (A3) comprises at least the step of:
(B1) filtering the at least one isocyanate from step (A2) through a filter having a permeability of 5 to 1001/(m²·min) under standard conditions, Δp=1 bar, H₂O, 20° C., to obtain the at least one isocyanate, or step (A3) comprises at least the steps of:
(B2) filtering the at least one isocyanate from step (A2) through a filter having a permeability of 5 to 1000 l/(m²·min) under standard conditions, Δp=1 bar, H₂O, 20° C., and
(C) filtering the at least one isocyanate through a filter having a maximum pore size of 0.02 to 10 μm,
   wherein the sequence of steps may be (A1), (A2) and (B1) or (A1), (A2), (B2) and (C) or (A1), (A2), (C) and (B2), to obtain the at least one isocyanate.

Steps (B1), (B2) and (C) are described in detail hereinabove.

The present invention relates in particular to the process according to the invention, wherein in step (A3) the at least one isocyanate is distilled before the filtering, i.e. before the steps (B1) or (B2) and (C). Such a distillation is carried out by processes known to those skilled in the art and the purifying distillation is carried out for example at a bottoms temperature in the range from 120° C. to 185° C. and a pressure of 1 to 100 mbar(a).

The process according to the invention affords the at least one isocyanate with a very low turbidity. The present invention therefore also relates to the at least one isocyanate obtainable, preferably obtained, by the process according to the invention.

The present invention also relates to an isocyanate, preferably a diisocyanate, more preferably xylylene diisocyanate, in particular meta-xylylene diisocyanate and/or para-xylylene diisocyanate, having a turbidity of less than 0.35 NTU, particularly preferably 0.05 to 0.35 NTU, very particularly preferably 0.10 to 0.32 NTU, in each case determined according to DIN EN ISO 7027-1: 2016.

The present invention also relates to a polymer constructed from at least one diisocyanate according to the invention and at least one isocyanate-reactive compound selected from the group consisting of polythiols containing at least two thiol groups, hydroxythiols containing at least one hydroxyl and thiol group, and polyols containing at least two hydroxyl groups. It is also possible to use mixtures of different isocyanate-reactive compounds. The isocyanate-reactive compound is preferably a polythiol or a mixture of two or more polythiols.

Suitable polythiols include for example methanedithiol, ethane-1,2-dithiol, propane-1,1-dithiol, propane-1,2-dithiol, propane-1,3-dithiol, propane-2,2-dithiol, butane-1,4-dithiol, butane-2,3-dithiol, pentane-1,5-dithiol, hexane-1,6-dithiol, propane-1,2,3-trithiol, cyclohexane-1,1-dithiol, cyclohexane-1,2-dithiol, 2,2-dimethylpropane-1,3-dithiol, 3,4-dimethoxybutane-1,2-dithiol and 2-methylcyclohexane-2,3-dithiol, thioether group-containing polythiols, for example 2,4-dimercaptomethyl-1,5-dimercapto-3-thiapentane, 4-mercaptomethyl-1,8-dimercapto-3,6-dithiaoctane, 4,8-dimercaptomethyl-1,11-dimercapto-3,6,9-trithiaundecane, 4,7-dimercaptomethyl-1,11-dimercapto-3,6,9-trithiaundecane, 5,7-dimercaptomethyl-1,11-dimercapto-3,6,9-trithiaundecane, 4,5-bis(mercaptoethylthio)-1,10-dimercapto-3,8-dithiadecane, tetrakis(mercaptomethyl)methane, 1,1,3,3-tetrakis(mercaptomethylthio)propane, 1,1,5,5-tetrakis(mercaptomethylthio)-3-thiapentane, 1,1,6,6-tetrakis(mercaptomethylthio)-3,4-dithiahexane, 2-mercaptoethylthio-1,3-dimercaptopropane, 2,3-bis(mercaptoethylthio)-1-mercaptopropane, 2,2-bis(mercaptomethyl)-1,3-dimercaptopropane, bis(mercaptomethyl) sulfide, bis(mercaptomethyl) disulfide, bis(mercaptoethyl) sulfide, bis(mercaptoethyl) disulfide, bis(mercaptopropyl) sulfide, bis(mercaptopropyl) disulfide, bis(mercaptomethylthio)methane, tris(mercaptomethylthio)methane, bis(mercaptoethylthio)methane, tris(mercaptoethylthio)methane, bis(mercaptopropylthio)methane, 1,2-bis(mercaptomethylthio)ethane, 1,2-bis(mercaptoethylthio)ethane, 2-mercaptoethylthio)ethane, 1,3-bis(mercaptomethylthio)propane, 1,3-bis(mercaptopropylthio)propane, 1,2,3-tris(mercaptomethylthio)propane, 1,2,3-tris(mercaptoethylthio)propane, 1,2,3-tris(mercaptopropylthio)propane, tetrakis(mercaptomethylthio)methane, tetrakis(mercaptoethylthiomethyl)methane, tetrakis(mercaptopropylthiomethyl)methane, 2,5-dimercapto-1,4-dithiane, 2,5-bis(mercaptomethyl)-1,4-dithiane and its oligomers obtainable according to JP-A 07118263, 1,5-bis(mercaptopropyl)-1,4-dithiane, 1,5-bis(2-mercaptoethylthiomethyl)-1,4-dithiane, 2-mercaptomethyl-6-mercapto-1,4-dithiacycloheptane, 2,4,6-trimercapto-1,3,5-trithiane, 2,4,6-trimercaptomethyl-1,3,5-trithiane and 2-(3-bis(mercaptomethyl)-2-thiapropyl)-1,3-dithiolane, polyesterthiols, for example ethylene glycol bis(2-mercaptoacetate), ethylene glycol bis(3-mercaptopropionate), diethylene glycol 2-mercaptoacetate, diethylene glycol 3-mercaptopropionate, 2,3-dimercapto-1-propanol 3-mercaptopropionate, 3-mercaptopropane-1,2-diol bis(2-mercaptoacetate), 3-mercaptopropane-1,2-diol bis(3-mercaptopropionate), trimethylolpropane tris(2-mercaptoacetate), trimethylolpropane tris(3-mercaptopropionate), trimethylolethane tris(2-mercaptoacetate), trimethylolethane tris(3-mercaptopropionate), pentaerythritol tetrakis(2-mercaptoacetate), pentaerythritol tetrakis(3-mercaptopropionate), glycerol tris(2-mercaptoacetate), glycerol tris(3-mercaptopropionate), cyclohexane-1,4-diol bis(2-mercaptoacetate), cyclohexane-1,4-diol bis(3-mercaptopropionate), hydroxymethyl sulfide bis(2-mercaptoacetate), hydroxymethyl sulfide bis(3-mercaptopropionate), hydroxyethyl sulfide 2-mercaptoacetate, hydroxyethyl sulfide 3-mercaptopropionate, hydroxymethyl disulfide 2-mercaptoacetate, hydroxymethyl disulfide 3-mercaptopropionate, (2-mercaptoethyl ester) thioglycolate and bis(2-mercaptoethyl ester) thiodipropionate and aromatic thio compounds, for example 1,2-dimercaptobenzene, 1,3-dimercaptobenzene, 1,4-dimercaptobenzene, 1,2-bis(mercaptomethyl)benzene, 1,4-bis(mercaptomethyl)benzene, 1,2-bis(mercaptoethyl)benzene, 1,4-bis(mercaptoethyl)benzene, 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(mercaptoethyl)benzene, 1,3,5-tris(mercaptoethyl)benzene, 1,2,4-tris(mercaptoethyl)benzene, toluene-2,5-dithiol, toluene-3,4-dithiol, naphthalene-1,4-dithiol, naphthalene-1,5-dithiol, naphthalene-2,6-dithiol, naphthalene-2,7-dithiol, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(mercaptoethyl)benzene, 1,2,3,5-tetrakis(mercaptoethyl)benzene, 1,2,4,5-tetrakis(mercaptoethyl)benzene, 2,2′-dimercaptobiphenyl, 4,4′-dimercaptobiphenyl or mixtures thereof.

It is preferable when the polythiol is selected from 4-mercaptomethyl-1,8-dimercapto-3,6-dithiaoctane, 2,5-bismercaptomethyl-1,4-dithiane, 1,1,3,3-tetrakis(mercaptomethylthio)propane, 5,7-dimercaptomethyl-1,11-dimercapto-3,6,9-trithiaundecane, 4,7-dimercaptomethyl-1,11-dimercapto-3,6,9-trithiaundecane, 4,8-dimercaptomethyl-1,11-dimercapto-3,6,9-trithiaundecane, trimethylolpropane tris(3-mercaptopropionate), trimethylolethane tris(2-mercaptoacetate), pentaerythritol tetrakis(2-mercaptoacetate), pentaerythritol tetrakis(3-mercaptopropionate) or mixtures thereof.

The invention also relates to a polythiourethane constructed from at least one purified isocyanate according to the invention and at least one polythiol or a thiol component B).

Aside from the thiol component B) the composition according to the invention may also contain other components typically reacted with polyisocyanates. These are in particular the customary polyetherpolyols, polyesterpolyols, polyetherpolyesterpolyols, polythioetherpolyols, polymer-modified polyetherpolyols, graft polyetherpolyols, in particular those based on styrene and/or acrylonitrile, polyetherpolyamines, hydroxyl-containing polyacetals and/or hydroxyl-containing aliphatic polycarbonates known from polyurethane chemistry which typically have a weight-average molecular weight of 106 to 12 000 g/mol, preferably 250 to 8000 g/mol. A broad overview of suitable coreactants B) may be found for example in N. Adam et al.: “Polyurethanes”, Ullmann's Encyclopedia of Industrial Chemistry, Electronic Release, 7th ed., chap. 3.2-3.4, Wiley-VCH, Weinheim 2005.

The present invention also relates to the use of the isocyanate according to the invention for producing polyurethanes and/or polythiourethanes for optical components. Examples of optical components are for example optical lenses, spectacle lenses or optoelectronic components such as light emitting diodes.

The present invention thus further also relates to an optical component, preferably a lens or a spectacle lens, containing at least one polyurethane according to the invention and/or at least one polythiourethane according to the invention.

### EXAMPLES

According to the invention the permeability of the employed filters is to be understood as meaning the amount of pure water passing through the filter layer per unit time and area under standard conditions, i.e. 20° C. and 1 bar pressure difference. Permeability is determined using ultrapure water. The filter or a representative sample of the filter material is subjected to ultrapure water at 20° C. and the desired pressure difference of 1 bar between the inflow side and the filtrate side is established. The time and the amount of filtrate generated in this time are measured, and taking into account the known area of the filter the permeability may be calculated according to the formula:

Permeability=amount of filtrate in liters/(filter area in m²×time in min)

A suitable apparatus for the measurement is described for example in VDI Guideline 2762, sheet 2, pages 6-8. What is relevant for the process according to the invention is the initial permeability of the employed filter before use.

The maximum pore size of the employed filters is determined according to ASTM F316-03 2011 for flexible filters, for example membrane filters, and according to ASTM E218-99 2011 for rigid filters, for example glass or ceramic filters.

The turbidity of the isocyanates is determined according to DIN EN ISO 7027-1: 2016-11, wherein the isocyanate is treated as an aqueous medium according to this DIN standard.

Reaction of meta-xylylenediamine (meta-XDA) with phosgene in the gas phase affords meta-xylylene diisocyanate (meta-XDI). The crude product is purified by distillation at 170° C. and a pressure of 15 mbar(a). The thus obtained meta-XDI (“starting solution”, experiment 9 in table 1) has a turbidity of 25.4 NTU determined according to DIN EN ISO 7027-1:2016-11. This starting solution is filtered according to experiments 1, 2, 3, 4, 5, V6, 7 and V8 recited in table 1.

The results are shown in table 1. The inventive experiments 1, 2, 3, 4, 5 and 7 achieve turbidity values desired according to the invention of ≤0.35 NTU and so polyurethanes and/or polythiourethanes produced with this meta-XDI meet the stringent requirements for use in optical lenses whereas the comparative experiments V6 and V8 result in disadvantageously high turbidity values and so polyurethanes and/or polythiourethanes produced therefrom are not suitable for use in optical lenses.

TABLE 1

| | | | | | | | | | 9 |
|---|---|---|---|---|---|---|---|---|---|
| | | | | | | | | | Starting |
| Number | 1 | 2 | 3 | 4 | 5 | V6 | 7 | V8 | solution |
| Permeability [l/m² · min] | 29 | 29 | 100 | 100 | 146 | 146 | 925 | 925 | — |
| in the 1st filtration stage | | | | | | | | | |
| Maximum pore size [μm] | — | 0.2 | 0.2 | 0.2 | 0.2 | — | 0.2 | — | — |
| in the 2nd filtration stage | | | | | | | | | |
| Turbidity [NTU]¹ | 0.27 | 0.23 | 0.32 | 0.29 | 0.26 | 14.7 | 0.32 | 20.8 | 25.4 |

V comparative experiment
— not performed
¹determined according to DIN EN ISO 7027-1: 2016

1. A process for purifying at least one isocyanate selected from the group consisting of aliphatic, cycloaliphatic, araliphatic isocyanates, and mixtures thereof, the process comprising the steps of:
(A) providing the at least one isocyanate,
(B1) filtering the at least one isocyanate from step (A) through a filter having a permeability of 5 to 1001/(m²·min) under standard conditions of Δp=1 bar, H₂O, 20° C., to obtain at least one purified isocyanate,
or comprising the steps of:
(A) providing the at least one isocyanate,
(B2) filtering the at least one isocyanate from step (A) through a filter having a permeability of 5 to 1000 l/(m²·min) under standard conditions of Δp=1 bar, H₂O, 20° C., and
(C) filtering the at least one isocyanate through a filter having a maximum pore size of 0.02 to 10 μm,
wherein the sequence of steps is (A), (B2), (C) or (A), (C), (B2), to obtain at least one purified isocyanate.

2. The process as claimed in claim 1, wherein the at least one purified isocyanate, optionally after one or more intermediate steps, is employed in a further process step for synthesis of one selected from the group consisting of polyurethanes and polythiourethanes for optical applications.

3. The process as claimed in claim 1, wherein the at least one isocyanate is selected from the group consisting of bis(isocyanatomethyl)bicyclo[2.2.1]heptane (NBDI), 2,5 bis(isocyanatomethyl)bicyclo[2.2.1]heptane (2,5-NBDI), 2,6-bis(isocyanatomethyl)bicyclo[2.2.1]heptane (2,6-NBDI), pentamethylene diisocyanate, 1,3-bis(isocyanatomethyl)cyclohexane (H₆XDI), xylylene diisocyanate, meta-xylylene diisocyanate, para-xylylene diisocyanate, and mixtures thereof.

4. The process as claimed in claim 1, wherein step (A) comprises distilling the at least one isocyanate, before steps (B1) or (B2).

5. The process as claimed in claim 1, wherein the filter employed in step (B1) or (B2) contains at least one material selected from the group consisting of natural fibers, synthetic polymers, perlite, kieselguhr, and mixtures thereof.

6. The process as claimed in claim 1, wherein step (B1) is operated at a specific filtrate flow of 50 to 1500 kg/(m²·h).

7. A process for producing at least one isocyanate selected from the group consisting of aliphatic, cycloaliphatic, araliphatic isocyanates, and mixtures thereof, the process comprising the steps of:
(A1) providing at least one amine corresponding to the isocyanate to be produced,
(A2) reacting the at least one amine from step (A1) with phosgene or a derivative thereof to obtain the at least one isocyanate and
(A3) purifying the at least one isocyanate from step (A2), wherein step (A3) comprises the step of:
(B1) filtering the at least one isocyanate from step (A2) through a filter having a permeability of 5 to 1001/(m²·min) under standard conditions of Δp=1 bar, H₂O, 20° C., to obtain the at least one isocyanate,
or step (A3) comprises the steps of:
(B2) filtering the at least one isocyanate from step (A2) through a filter having a permeability of 5 to 1000 l/(m²·min) under standard conditions of Δp=1 bar, H₂O, 20° C., and
(C) filtering the at least one isocyanate through a filter having a maximum pore size of 0.02 to 10 μm,
wherein the sequence of steps is one selected from the group consisting of (A1), (A2) and (B1); (A1), (A2), (B2) and (C); (A1), (A2), (C) and (B2), to obtain the at least one isocyanate.

8. The process as claimed in claim 7, wherein step (A2) (A2) is carried out at a temperature of 5° C. to 500° C. and a pressure of 0.5 to 10 bar(a).

9. The process as claimed in claim 7, wherein in step (A3) the at least one isocyanate is distilled before filtering.

10. An isocyanate obtained by the process as claimed in claim 1.

11. An isocyanate obtained by the process as claimed in claim 1, wherein the isocyanate has a turbidity of less than 0.35 NTU determined according to DIN EN ISO 7027-1: 2016.

12. A polyurethane comprising at least one isocyanate as claimed in claim 10 and at least one polyol.

13. A polythiourethane comprising at least one isocyanate as claimed in claim 10 and at least one polythiol.

14. In a process for producing one of polyurethanes and polythiourethanes for optical applications, the improvement comprising including the isocyanate as claimed in claim 10.

15. An optical component containing at least one polyurethane as claimed in claim 12.

16. An optical component containing at least one polyurethane as claimed in claim 13.

17. The process as claimed in claim 1, wherein step (B1) is operated at a specific filtrate flow of 100 to 1000 kg/(m²·h).

18. The process as claimed in claim 1, wherein step (B1) is operated at a specific filtrate flow of 200 to 500 kg/(m²·h).
